(19) United States
(12) Patent Application Publication
(10) Pub. No.: US 2016/0067081 A1
(43) Pub. Date: Mar. 10, 2016

(54) MANDIBULAR ADVANCEMENT DEVICE WITH POSITIVE AIR PRESSURE

(71) Applicant: Dennis Peter Sorresso, Orange Park, FL (US)

(72) Inventor: Dennis Peter Sorresso, Orange Park, FL (US)

(21) Appl. No.: 14/802,031

(22) Filed: Jul. 17, 2015

Related U.S. Application Data
(60) Provisional application No. 62/048,336, filed on Sep. 10, 2014.

(51) Int. Cl. A61F 5/56; A61M 16/04; A61M 16/08; A61M 16/00

(52) U.S. Cl. CPC A61F 5/566; A61M 16/0057; A61M 16/049; A61M 16/0875

(57) ABSTRACT

A dental appliance for the treatment of sleep disordered breathing. It addresses vibration and collapse of the soft palate, obstruction of the upper airway by the tongue and problems caused by retro/or micro-gnathia. The device includes upper trays, lower trays, and channels for providing positive or negative air pressure through the device.

### CROSS-REFERENCE TO RELATED APPLICATION

This application claims the benefit of priority of U.S. provisional application No. 62/048,336, filed Sep. 10, 2014, the contents of which are herein incorporated by reference.

### BACKGROUND OF THE INVENTION

The present invention relates to mandibular advancement devices and, more particularly, to a mandibular advancement device with positive air pressure.

Obstructive sleep apnea syndrome many times does not respond to positive airway pressure or in certain circumstances, excessive pressures are required. Current mandibular advancement devices are limited to mild to moderate cases of obstructive sleep apnea syndrome. Current mandibular advancement devices can lead to bite change, jaw pain and shifting of teeth.

As can be seen, there is a need for a mandibular advancement device with positive air pressure, that addresses vibration and collapse of the soft palate, obstruction of the upper airway by the tongue and problems caused by retro/or micro-gnathia.

### SUMMARY OF THE INVENTION

In one aspect of the invention, a mandibular advancement device with positive air pressure includes, an upper tray and a lower tray, wherein at least one of the upper tray and the lower tray comprises a plurality of hollow channels; at least two straps, wherein the straps connect the upper tray to the lower tray; and at least one airway tube in fluid communication with said plurality of hollow channels.

In another aspect of the invention, the at least one airway tube further comprising multiple branches, wherein each branch enters through one of the plurality of hollow channels.

A further aspect of the invention includes, adjustable length straps and straps that are hingedly connected to a post extending from an aft portion of the upper tray and lower tray.

In certain embodiments, the hollow channels have openings extending to an inner surface of the at least one of the upper tray and said lower tray. Further, the hollow channels may also comprise an opening at an aft end of the at least one of said upper tray and said lower tray.

An aspect of the invention also includes a mandibular advancement device with positive air pressure having an upper tray and a lower tray, with a hollow channel defined in at least one of the upper tray and said lower tray; at least two straps connect the upper tray to the lower tray; and at least one airway tube in fluid communication with said hollow channel. The hollow channel has a plurality of openings to an inner surface of the tray of mandibular advancement device. The hollow channel may also have an opening at an aft end of said tray and may further include a hollow channel is defined in each of said upper tray and said lower tray.

In a further aspect, the mandibular advancement device is provided a first airway tube in fluid communication with a hollow channel in the upper tray, and a second airway tube is in fluid communication with the hollow channel in the lower tray.

In another instance of the present invention, the upper tray and lower tray may also be formed to an impression of a patient's teeth.

These and other features, aspects and advantages of the present invention will become better understood with reference to the following drawings, description and claims.

### BRIEF DESCRIPTION OF THE DRAWINGS

FIG. 1: is a perspective view of an embodiment of a mandibular advancement device

FIG. 2: is an exploded view of the an embodiment of a mandibular advancement device

FIG. 3: is a section view of the invention, taken along line 3-3 in FIG. 1, illustrating the exemplary movement of negative airflow through the lower mouthpiece 10 channels

FIG. 4: is a section view of the invention, taken along line 4-4 in FIG. 1, illustrating the exemplary movement of positive airflow through the upper mouthpiece 10 channels

FIG. 5: is a side view of the invention, illustrating its placement on the user's teeth 78

FIG. 6: is a side view of the invention, illustration the adjustment of strap 14

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a mandibular advancement device with positive air pressure comprising: an upper tray and a lower tray, wherein at least one of the upper tray and the lower tray comprises a plurality of hollow channels; at least two adjustable components, wherein the adjustable components connect the upper tray to the lower tray; and an air tube comprising multiple branches, wherein each branch enters through one of the plurality of hollow channels.

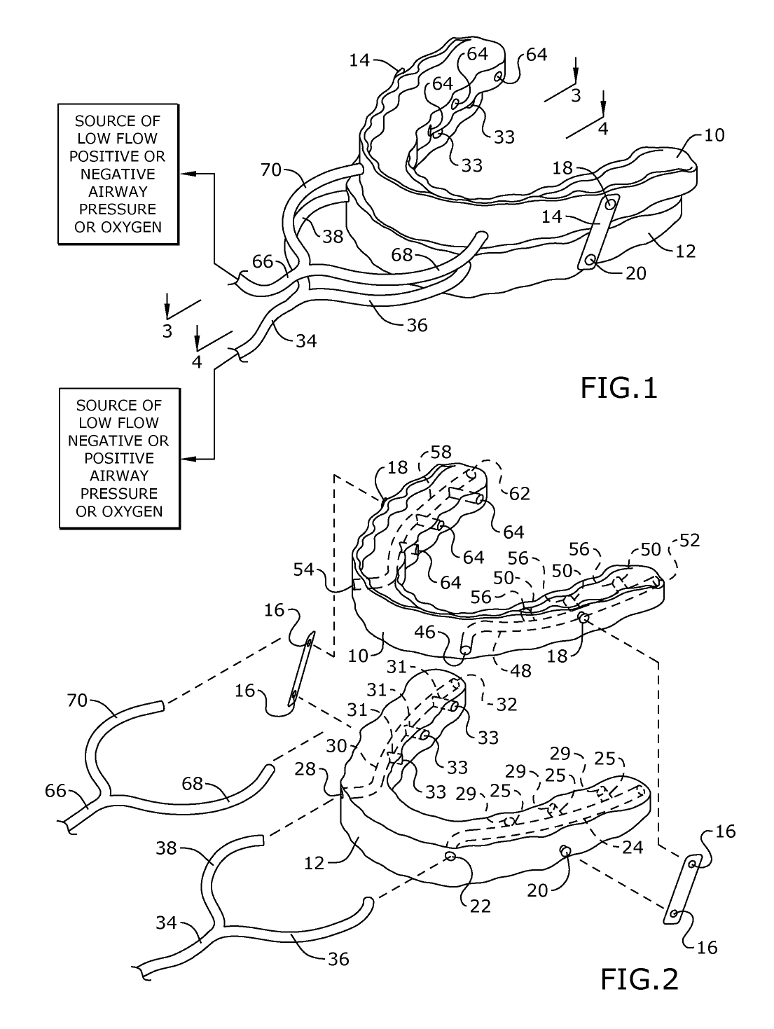

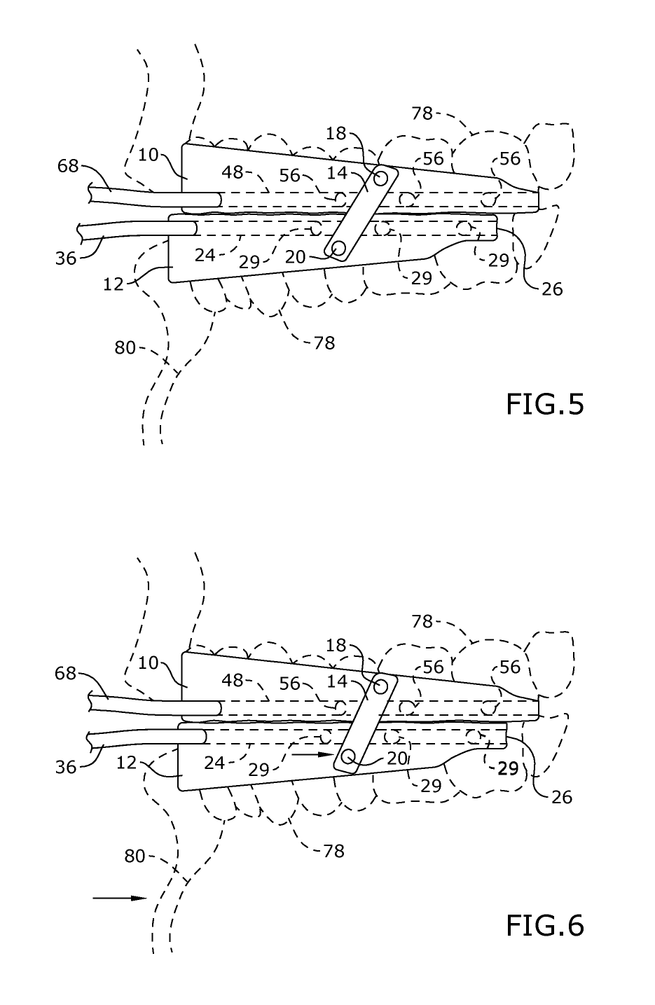

As is illustrated in FIGS. 1 through 6, a device may include an upper mouthpiece 10 and a lower mouthpiece 14 each having a front end and two back ends. The upper mouthpiece 10 and the lower mouthpiece 12 may be connected by at least two straps 14. The straps 14 may each include holes 16 at the ends thereof, which are pivotally attached to an upper mouthpiece strap post 18 and a lower mouthpiece strap post 20 extending from an outer surface and proximal to an aft end of the respective upper and lower mouthpieces. The straps 14 may be placed along approximately opposite sides of the upper mouthpiece 10 and the lower mouthpiece 12. The upper mouthpiece 10, the lower mouthpiece 12, and the at least two straps 14 may function as a mandibular advancement portion of the device.

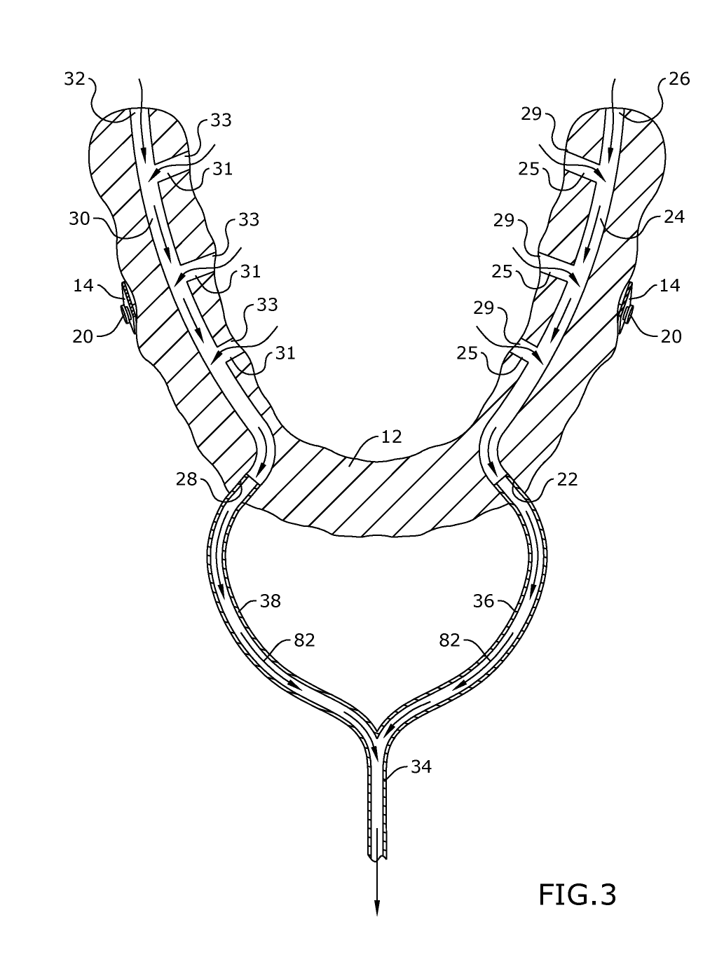

The lower mouthpiece 12 may have at least two hollow channels, a left lower channel 24 and a right lower channel 30 that each run from the front end to one of the back ends of the lower mouthpiece 12. As may be seen in reference to FIGS. 2 and 3, left lower channel 24 extends between a left lower front channel opening 22 to a left lower rear channel opening 26. A plurality of left lower side channels 25 may extend from an left lower side channel openings 26 on an inner surface of the lower mouthpiece 12 and left lower channel 24. In like manner, the right lower channel 30 extends from a right lower front channel opening 28 at a front end of the lower mouthpiece 12 to a right lower rear channel opening 32. Lower mouthpiece 12 may include a plurality of right lower side channels 31 extending from right lower side channel openings 33 on an inner surface of the lower mouthpiece 12 and the right lower channel 30.

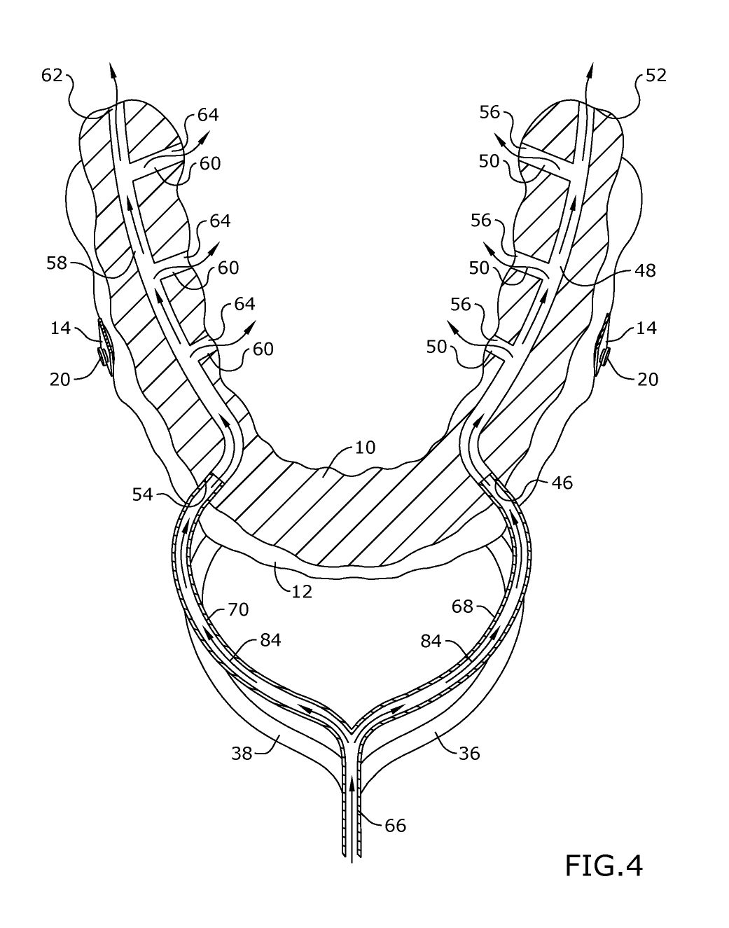

The upper mouthpiece 10, may have at least two hollow channels, a left upper channel 48 and a right upper channel 58 that each run from the front end to one of the back ends of the upper mouthpiece 10. As may be seen in reference to FIGS. 2 and 4, left upper channel 48 extends between a left upper front channel opening 46 to a left upper rear channel opening 52. A plurality of left upper side channels 50 may extend from a left upper side channel opening 56 on an inner surface of the upper mouthpiece 10 and left upper channel 48. In like manner, the right upper channel 58 extends from a right upper front channel opening 54 at a front end of the upper mouthpiece 10 to a right upper rear channel opening 62. Upper mouthpiece 10 may include a plurality of right upper side channels 60 extending from right upper side channel openings 64 on an inner surface of the upper mouthpiece 10 and the right upper channel 58.

As best seen in reference to FIGS. 1 and 3, a lower central airway tube 34 may have branches defining a left lower airway tube 36 and right lower airway tube 38 that each may operatively connect the lower central airway tube 34 with the respective left and right channels 24, 30 via their associated channel openings 22, 28.

As best seen in reference to FIGS. 1 and 4, an upper central airway tube 66 may have branches defining a left upper airway tube 68 and right upper airway tube 70 that each may operatively connect the upper central airway tube 66 with the respective left and right channels 48, 58 via their associated channel openings 46, 54.

As may be seen with reference to FIGS. 3 and 4, a source of low flow positive airway pressure 84 or oxygen may send pressurized air 84, and/or supplemental oxygen or the like through the central airway tube 66 to the hypopharynx. Similarly a source of low flow negative pressure air 82 may be applied to the lower central airway tube 34 to evacuate air from the hypopharynx. As will be appreciated and as illustrated in reference to FIG. 1, connections between the pressurized 84 or negative airflow 82 sources and the respective upper 66 or lower 34 central airway tubes may be reversed.

According to another aspect of the mandibular advancement device, the upper mouthpiece 10 and the lower mouthpiece 12, may extend the mandible 80 forward while the air channels may force positive pressure 84 toward the hypopharynx. Both the advancement of the mandible 80 and the addition of the positive pressure 84 may be included within one device.

The device may be produced by taking mold templates of an individual's top and bottom teeth 78. In certain embodiments, a generic sized mold may be made for temporary applications. The upper mouthpiece 10 and the lower mouthpiece 12 may be linked by a mechanism that may allow adjustment to move the mandible 80 forward. In certain embodiments, the lower mouthpiece 12, the upper mouthpiece 10, or both the upper mouthpiece 10 and the lower mouthpiece 12 may include the hollow channels 24, 25, 30, 31, 48, 50, 58, and 60 that may allow air to pass through the mandibular advancement device and exit at the back end positioned toward the back of the mouth (i.e., oropharynx/hypopharynx). The hinged straps 14 may be any number of variations or simply a component to link the upper mouthpiece 10 and the lower mouthpiece 12 of the mandibular advancement device. In certain embodiments, a tongue retainer may be added to further improve the ability of the device to keep the airway (i.e., oropharynx/hypopharynx) open.

The straps 14 between the upper mouthpiece 10 and the lower mouthpiece 12 of the mouthpiece (i.e., mandibular advancement device) may be any number of variations in order to link and set the upper mouthpiece 10 and the lower mouthpiece 12 together; and adjustable to move the lower mouthpiece 12 forward. The air channels for one or both of the upper mouthpiece 10 and the lower mouthpiece 12 may be arranged in a variety of number and configurations in order to allow air to enter and exit the upper mouthpiece 10 and the lower mouthpiece 12.

The channels in the upper and lower mouthpieces can be used together or separately. Moreover, negative and positive pressure or supplemental oxygen can be used interchangeably and at the same time in both the upper 10 and lower 12 mouth pieces. For example, negative pressure can be utilized in the lower mouth piece 12 for the purpose of tongue retention. In this case, the suction provided at the left and right side openings 29, 31 will retain the tongue in position against an inner surface of the lower tray 12. Simultaneously, positive pressure and/or supplemental oxygen can be delivered via the upper tray 10 to ‘push’ the soft palate and hypo-pharynx open. The upper and bottom trays 10, 12 can be used separately in order to utilize only positive or negative pressure and/or supplemental oxygen. Or they can be connected by an adjustment strut to also engage for mandibular advancement.

By utilizing mandibular advancement with positive and/or negative airway pressure with or without supplemental oxygen the user will treat sleep disordered breathing. Additionally, the device can be used in the outpatient setting to treat chronic disorder. The device can be used in the inpatient setting to treat acute respiratory failure. Similarly, the device can be used in the diagnostic setting to determine the best settings to treat a respiratory disorder.

The device may be designed to address the treatment for sleep disordered breathing; the more common known or treated being Obstructive Sleep Apnea Syndrome, acute respiratory failure or the like. Specifically the device may keep the upper airway (i.e., oral & hypo-pharynx) open while asleep or awake and receive respiratory/ventilator assist.

It should be understood, of course, that the foregoing relates to exemplary embodiments of the invention and that modifications may be made without departing from the spirit and scope of the invention as set forth in the following claims.

What is claimed is:

1. A mandibular advancement device with positive air pressure comprising:
an upper tray and a lower tray, wherein at least one of the upper tray and the lower tray comprises a plurality of hollow channels;
at least two straps, wherein the straps connect the upper tray to the lower tray; and
at least one airway tube in fluid communication with said plurality of hollow channels.

2. The mandibular advancement device of claim 1, said at least one airway tube further comprising multiple branches, wherein each branch enters through one of the plurality of hollow channels.

3. The mandibular advancement device of claim 1, wherein said straps are adjustable in a length.

4. The mandibular advancement device of claim 1, wherein said straps are hingedly connected to a post extending from an aft portion of said upper tray and lower tray.

5. The mandibular advancement device of claim 1, wherein, said hollow channels have openings extending to an inner surface of said at least one of said upper tray and said lower tray.

6. The mandibular advancement device of claim 5, wherein said hollow channels further comprise an opening at an aft end of said at least one of said upper tray and said lower tray.

7. A mandibular advancement device with positive air pressure comprising:
an upper tray and a lower tray, with a hollow channel defined in at least one of said upper tray and said lower tray;
at least two straps, wherein the straps connect the upper tray to the lower tray; and
at least one airway tube in fluid communication with said hollow channel.

8. The mandibular advancement device of claim 7, wherein said hollow channel has a plurality of openings to an inner surface of said tray.

9. The mandibular advancement device of claim 8, wherein said hollow channel has an opening at an aft end of said tray.

10. The mandibular advancement device of claim 9, wherein said hollow channel is defined in each of said upper tray and said lower tray.

11. The mandibular advancement device of claim 10, wherein a first airway tube is in fluid communication with a hollow channel in said upper tray, and a second airway tube is in fluid communication with said hollow channel in said lower tray.

12. The mandibular advancement device of claim 7, wherein said upper tray and said lower tray are formed to an impression of a patient's teeth.
